(19) United States
(12) Patent Application Publication
(10) Pub. No.: US 2010/0234385 A1
(43) Pub. Date: Sep. 16, 2010

(54) Compsition Containing Biopterin and Method for Using The Same

(75) Inventors: Hiroyuki Hasegawa, Uenohara-Shi (JP); Keiko Sawabe, Uenohara-shi (JP); Ichiro Shimizu, Osaka-shi (JP); Yasuhiro Ikenaka, Kobe-shi (JP)

Correspondence Address: BIRCH STEWART KOLASCH & BIRCH, PO BOX 747, FALLS CHURCH, VA, 22040-0747, US

(73) Assignee: Kaneka Corporation, Osaka-shi (JP)

(21) Appl. No.: 12/225,307

(22) PCT Filed: Mar. 15, 2007
(86) PCT No.: PCT/JP2007/055184

§ 371 Date: Dec. 18, 2008

(30) Foreign Application Priority Data
Mar. 20, 2006 (JP) 2006-076180

(51) Int. Cl. A61K 31/519; C07D 475/04; A61P 17/16; A61P 25/24; A61P 9/10; A61P 9/12; A61P 25/28; A61P 25/16; A61P 25/18; A61P 3/06; A61P 3/02

(52) U.S. Cl. 514/249; 544/258

(57) ABSTRACT

It is thought that applications and use of tetrahydrobiopterin (BH4), which is used as a pharmaceutical, will expand due to its superior efficacy. However, in addition to BH4 being extremely expensive, since it is unstable with respect to oxidation, applications other than pharmaceuticals are considered to be difficult. Further, it is difficult for BH4 to permeate through a blood-brain barrier, so that BH4 concentrations in the brain tend not to increase under the present conditions. Biopterins are oxidized form of BH4, stable to oxidation, easy to handle, and can be produced inexpensively. The inventors found that administration of biopterin can be expected to demonstrate effects equal to or greater than those of administration of BH4. On the basis of the result, it was first clarified that administration of a composition containing biopterin causes increase in BH4 concentration in the body (especially BH4 concentration in the brain, which is difficult to be increased by BH4 administration) to demonstrate sufficient actions.

### TECHNICAL FIELD

The present invention relates to a composition for using biopterin in pharmaceuticals, functional foods, supplements, foods, veterinary drugs, animals feeds, cosmetics and the like, and to method of using the same.

### BACKGROUND

In the present description, “biopterin” refers to 6-(L-erythro-1,2-dihydroxypropyl)-pterin or 7-(L-erythro-1,2-dihydroxypropyl)-pterin. This biopterin, including L-erythro-5,6,7,8-tetrahydrobiopterin (abbreviated as “BH4”) and the oxidized form of BH4 in the form of L-erythro-7,8-dihydrobiopterin (abbreviated as “BH2”), are generically referred to as “biopterins”.

Biopterin was first isolated from human urine as a growth factor of Trypanosoma by Petterson et al. in 1955 (Non-Patent Document 1). Biopterins are known to present in comparative large amounts in, for example, various organs, the skin of certain species of reptiles, amphibians and birds and the eyes of fruit flies.

In addition, BH4 acts as an essential coenzyme of hydroxylase reactions of aromatic amino acids in the first step of the synthesis of mammalian neurotransmitters such as serotonin and dopamine. In addition, BH4 is also known to be involved as a cofactor of nitrogen monoxide synthase involved in vasoconstriction and the like.

Known examples of methods for producing biopterins include biological methods and chemical synthesis methods. Although examples of biological methods include methods for extracting from the organisms described above and methods using microorganisms (Patent Document 1, Patent Document 2) or biosynthases (Patent Document 3), all of these methods have a considerable lack of productivity and are currently not used practically.

More recently, although a considerable level of productivity has been achieved with Escherichia coli by making use of genetic recombination techniques (Patent Document 4), there have yet to be reports indicating that biopterins have been produced practically using this technique. Chemical synthesis methods use practical methods for organically synthesizing from sugars such as rhamnose, and these methods are used for the industrial production of BH4 for use in pharmaceuticals.

In Japan, BH4 is used as a therapeutic drug for diseases originating in genetic deficiencies such as hyperphenylalaninemia caused by a deficiency of dihydrobiopterin synthetase or dihydropteridine reductase. In the US, BH4 is undergoing clinical studies for use a therapeutic drug for phenylketonuria and vascular diseases.

Patent Document 1: Japanese Kohyo Publication No. H05-33989

Patent Document 2: Japanese Kohyo Publication No. H05-33990

Patent Document 3: Japanese Kokai Publication No. H04-82888

Patent Document 4: WO 2002-018587

Non-Patent Document 1: Petterson, E. L. et al., J. Am. Chem. Soc., 77, 3167-3168 (1955)

Although biopterins are known to be present in foods such as royal jelly and mammalian milk, since their content therein is extremely low, consumption of such foods in amounts that would allow the functions of biopterins to be demonstrated is not realistic. Although biopterins have conventionally been known to be intermediates during chemical synthesis of BH4, or in the body, be excreted in urine or contained in milk, there have been few attempts made to elucidate their physiological role and action. The action of biopterin in humans and animals is still unknown, and a description of the functions thereof cannot be found in the literature.

On the other hand, BH4, which is the only biopterin produced industrially, is used as a pharmaceutical, it is thought that its applications and use will expand due to its superior efficacy, and it is also expected to demonstrate effects as a functional food. However, in addition to BH4 being extremely expensive, since it is unstable with respect to oxidation, applications other than pharmaceuticals are currently considered to be difficult.

As a result of conducting extensive studies, the inventors of the present invention found that administration of biopterin can be expected to demonstrate effects equal to or greater than those of administration of BH4. Namely, an examination of pharmacokinetics following administration of biopterin to mice and other mammals unexpectedly revealed that BH4 concentrations in the body increase, and in comparison with during administration of BH4, that the increase tended to be gradual and sustained. In addition, it was also found that BH4 concentrations in the brain, in which changes are not observed during BH4 administration, increase remarkably following administration of biopterin. Namely, the substance permeability of the blood-brain barrier is thought to be increased during administration of biopterin.

There have been no known findings thus far indicating that administered biopterin is taken up by the body or that BH4 concentration increases considerably as a result thereof, the enzyme reaction and so forth by which biopterin is reduced to BH4 is also unknown, and as such, findings relating to changes in BH4 concentrations in the body during administration of biopterin were first clarified by the present invention.

On the basis of these results, administration of a composition containing biopterin can be expected to cause a gradual and sustained increase in BH4 concentration in the body and demonstrate greater actions and effects than during administration of BH4. Namely, instead of the sudden increase in blood BH4 concentration observed in the case of administering BH4, in the case of adding biopterin, a comparatively mild and sustained increase is obtained, which in addition to being able to reduce adverse side effects such as the formation of active oxygen presumed to occur due to sudden increases in BH4 concentration, is thought to prolong time during which effects are sustained. In addition, the possibility is also strongly suggested of the administration of biopterin, which is thought to increase substance permeability of the blood-brain barrier to a greater degree than BH4, being dramatically effective as a therapeutic agent or preventive agent for conditions in which it is necessary to increase BH4 concentration is the brain, such as bipolar disorder or schizophrenia, infantile autism, attention deficit hyperactivity disorder (ADHD), chronic fatigue syndrome, Parkinson's disease or Alzheimer's disease.

Alternatively, there is also a high likelihood of biopterin being able to be produced inexpensively since it more stable with respect to oxidation and handled more easily than BH4.

Thus, a composition having effects equal to or greater than those of BH4 can be provided inexpensively and in a form that offers easily handling.

### SUMMARY OF THE INVENTION

Accordingly, the present invention provides the following:

(1) a biopterin composition which contains 100 μg or more of biopterin per 1 g of composition, and can be used as a pharmaceutical, functional food, supplement, food, cosmetic, veterinary drug, veterinary supplement or animal feed;
(2) the biopterin composition described in (1), containing 1 mg or more of biopterin per 1 g of composition;
(3) a biopterin used to increase the concentration of tetrahydrobiopterin in the body;
(4) the biopterin composition described in (1) and (2) or the biopterin described in (3), having a therapeutic effect against malignant phenylketonuria or Segawa disease (dopa responsive dystonia) caused by tetrahydrobiopterin deficiency, or against phenylketonuria associated with inadequate activity of phenylalanine hydroxylase caused by the mutation thereof;
(5) the biopterin composition described in (1) and (2) or the biopterin described in (3), having a protective action on vascular disorders caused by hypertension by promoting production of nitrogen monoxide in human or animal blood vessels and lowering blood pressure as a result of vasodilation due to the action of producing nitrogen monoxide;
(6) the biopterin composition described in (1) and (2) or the biopterin described in (3), having a protective action on vascular disorders caused by the formation of active oxygen by promoting production of nitrogen monoxide in human or animal blood vessels and eliminating active oxygen by the action of producing nitrogen monoxide;
(7) the biopterin composition described in (1) or (2) or the biopterin described in (3), having an effect of improving symptoms of bipolar disorder or schizophrenia, infantile autism, attention deficit hyperactivity disorder (ADHD), chronic fatigue syndrome, Parkinson's disease or Alzheimer's disease by promoting the formation of brain monoamines;
(8) the biopterin composition described in (1) or (2) or the biopterin described in (3), having an effect of improving symptoms of depression, fatigue, arteriosclerosis, hypertension, hypercholesterolemia or impairment of vascular endothelial function by smoking;
(9) the biopterin composition described in (1) or (2) or the biopterin described in (3), having an effect of improving symptoms of depression, fatigue, arteriosclerosis, hypertension or impairment of vascular endothelial function by hypercholesterolemia in animals;
(10) the biopterin composition described in (1) or (2) or the biopterin described in (3), having sunburn preventive or whitening effects by inhibiting dermal melanocytes;
(11) the biopterin composition described in any of (1) to (10), demonstrating an effect that is mild and has superior sustainability as a result of using as a partial or complete alternative to tetrahydrobiopterin during administration thereof;
(12) the biopterin composition according to any of claims 1 to 10, demonstrating a superior increase in intracerebral tetrahydrobiopterin concentration as a result of using as a partial or complete alternative to tetrahydrobiopterin during administration thereof;
(13) a pharmaceutical containing the biopterin composition described in (1) or (2) or the biopterin described in (3);
(14) a functional food, supplement or food containing the biopterin composition described in (1) or (2) or the biopterin described in 3);
(15) a veterinary drug, veterinary supplement or animal feed containing the biopterin composition described in (1) or (2) or the biopterin described in (3);
(16) a skin agent or cosmetic containing the biopterin composition described in (1) or (2) or the biopterin described in (3); and
(17) a use of biopterin for producing the biopterin composition described in (1) or (2).

The use of a composition containing biopterin, for which effects were found by the inventors of the present invention, allows biopterins, for which effects are expected to be demonstrated in the form of pharmaceuticals, functional foods, supplements, foods, veterinary drugs, animal feeds or cosmetics and the like, to be used stably, inexpensively and with ease of handling.

### DETAILED DESCRIPTION OF THE INVENTION

As previously described in the section entitled “Background Art”, in the present description, “biopterin” refers to 6-(L-erythro-1,2-dihydroxypropyl)-pterin or 7-(L-erythro-1,2-dihydroxypropyl)-pterin, and including L-erythro-5,6,7,8-tetrahydrobiopterin (abbreviated as “BH4”) and the oxidized form thereof in the form of L-erythro-7,8-dihydrobiopterin (abbreviated as “BH2”), are generically referred to as biopterins.

### 1. Biopterin

Although biopterin has conventionally been known to be an intermediate during chemical synthesis of BH4, be excreted from the body in the form of urine, or be contained in milk and the like, its physiological role and action have yet to be elucidated. The reason for this is that, since the active form is BH4, and the oxidized form thereof in the form of biopterin is contained in excrement and the like, it was thought to have been presumed to be inactive, thereby resulting in a lack of research on the activity thereof.

As will be subsequently described in the examples, although the finding elucidated by the inventors of the present invention that BH4 concentrations in the body are increased considerably by administration of biopterin strongly suggests the possibility that administered biopterin was taken up by the body and converted to BH4, there is also the possibility that biosynthesis of BH4 was considerably accelerated by the administered biopterin.

### 2. Effects of Biopterin

According to this finding, an embodiment of the present invention in the form of “biopterin” or “biopterin composition” (both of which are suitably referred to as “biopterin” for the sake of convenience) can be used with effects equal to or greater than those of BH4. BH4 has been clearly demonstrated or is expected to have the effects indicated below. Thus, “biopterin” of the embodiments is considered to at least have the effects indicated below.

First, examples of applications in which it is already used as a pharmaceutical or on which clinical studies are already underway include use as a therapeutic agent for malignant phenylketonuria or Segawa disease (dopa responsive dystonia) caused by a deficiency of tetrahydrobiopterin, phenylketonuria accompanying a lack of activity of phenylalanine hydroxylase, and peripheral arterial disease (PAD) or poorly controlled hypertension thought to be caused by a lack of activity of nitrogen monoxide synthase having tetrahydrobiopterin as a cofactor thereof. Moreover, since tetrahydrobiopterin reduces vascular endothelial disorders caused by active oxygen, the use of biopterin is also considered for the purpose of preventing tissue necrosis due to generation of active oxygen accompanying resumption of blood flow during treatment of embolisms for myocardial infarction and the like.

In addition, since it was determined in particular that continuous administration of biopterin is effective in increasing intracerebral BH4 concentration, expectations are being placed on the administration of biopterin, which is thought to increase substance permeability of the blood-brain barrier to a greater extent than administration of BH4 alone, as a therapeutic agent or supplement for diseases for which intracerebral BH4 concentration is suggested to be intimately involved, such as bipolar disorder or schizophrenia, Alzheimer's disease, infantile autism or attention deficit hyperactivity disorder (ADHD).

Since indications of biopterin are expected to cover an even wider range of neurological diseases such as depression or chronic fatigue syndrome, and diseases associated with vascular disorders such as arteriosclerosis or hypercholesterolemia, in the future, the deployment of biopterin as a supplement or functional food is being considered for the purpose of preventing these diseases. In addition, in consideration of the growing attention being placed on depression and other neurological diseases as well as vascular disorders in pets, there is also the possibility of biopterin being used in veterinary drugs, veterinary supplements and animal feeds in the future.

Alternatively, since biopterin has also been determined to have inhibitory effects on melanocytes in vitro, its use as a skin agent or cosmetic for whitening is also being considered.

### 3. Usage Forms

The dosage of BH4 for the above-mentioned indications is known to be about 10 mg per kg of body weight for treatment of phenylketonuria. In addition, efficacy has also been determined to be able to be obtained at the same dosage in clinical studies on peripheral arterial disease and poorly controlled hypertension. In consideration of these required amounts, the “biopterin composition” of the embodiments of the present invention is a composition that contains at least 100 μg, and more preferably at least 1 mg, of biopterin per 1 g of the composition, and can also be used in the form of a composition in which other active ingredients are also present. The biopterin composition has an action of increasing the concentration of tetrahydrobiopterin in the body of a human or animal in the case of having been ingested by a human or animal.

In addition, biopterin can be used in various forms such as tablets, capsules, powder, liquid or paste, and can be administered orally, by intravenous injection, by application to the skin or in the form of an ointment. Specific examples of these forms are described below. Preparation and so forth of the composition as described below can be carried out using known means by a person with ordinary skill in the art unless specifically stated otherwise.

### 3-1. Pharmaceutical and Veterinary Drug Compositions

In the case of a pharmaceutical composition or veterinary drug composition, the “biopterin composition” of the embodiments of the present invention can be prepared in various drug forms suitable for, for example, oral administration, intrarectal administration, intravenous injection, intramuscular injection, subcutaneous injection, intracutaneous injection, administration by instillation, intranasal administration, intrabuccal administration or suppositories, or administration through the skin by ointments or patches in the case of applications for external use.

In the case of preparing the “biopterin composition” of the embodiments, various pharmaceutically acceptable additives may be suitably added, examples of which include at least one type of carrier, diluent, vehicle, flow agent, binder, stabilizer, thickener or pH adjuster. In the case of using the “biopterin composition” in the form of capsules, a carrier such as lactose may also be added in addition to the stabilizers mentioned above.

The “biopterin composition” of the embodiments is able to promote permeation of an active ingredient into the skin by preparing and using in the form of a coating agent, ointment, cream or other preparation form suitable for topical application such as an aerosol, compress or poultice. In such cases, in addition to a pressure-sensitive adhesive, oily base and the like, an arbitrary component serving as a compounding agent ordinarily used in external skin preparations may be added, examples of which include surfactants, alcohols, moisture retention agents, thickeners, antiseptics, antioxidants, chelating agents, pH adjusters, fragrances, pigments, ultraviolet absorbers/light scattering agents, vitamins, amino acids and water. Furthermore, arbitrary components are not limited to these components.

### 3-2. Functional Foods, Supplements, Foods, Animal Feeds and Cosmetics

In addition to use as a pharmaceutical and veterinary drug, the “biopterin composition” discovered by the inventors of the present invention can also be used as a functional food, supplement, food, animal feed or cosmetic.

(Functional Foods)

In the case of a “functional food”, although oral administration is considered to be the primary form of administration, in the case of preparing the “biopterin composition” of the embodiments, preparation can be carried out by adding various types of additives allowed in the Food Sanitation Law and the like, and health food materials, nutritional supplement materials or vitamins and the like can also be contained. There are no particular limitations on these additives and materials, and examples thereof include vehicles, disintegration agents, lubricants, binders, coating agents, colorants, anti-aggregation agents, absorption promoters, dissolution assistants, stabilizers, health food materials, nutritional supplement materials, vitamins, fragrances, sweeteners, antiseptics, preservatives and antioxidants.

Examples of the vehicles include glucose, cornstarch, mannitol, crystalline cellulose, calcium phosphate and calcium sulfate.

Examples of the disintegration agents include starch, agar, calcium citrate, calcium carbonate, sodium bicarbonate, dextrin, crystalline cellulose, carboxymethyl cellulose and tragacanth.

Examples of the lubricants include components such as magnesium stearate, sodium stearate, stearic acid, calcium stearate, magnesium oleate, oleic acid, potassium oleate, caprylic acid, sodium stearyl fumarate and magnesium palmitate.

Examples of the binders include starch and derivatives thereof (such as alpha starch or dextrin), cellulose and derivatives thereof (such as ethyl cellulose, sodium carboxymethyl cellulose or hydroxypropyl methyl cellulose), gum arabic, tragacanth, gelatin, sugars (such as glucose or saccharose), ethanol and polyvinyl alcohol.

Examples of the coating agents include cellulose derivatives (such as hydroxypropyl cellulose, cellulose acetate phthalate or hydroxypropylmethyl cellulose phthalate), shellac, polyvinylpyrrolidone, polyvinylpyridines (such as poly-2-vinylpyridine or poly-2-vinyl-5-ethylpyridine), polyvinylacetyl diethylaminoacetate, polyvinyl alcohol phthalate and methacrylate-methacrylic acid copolymers.

Colorants for which use thereof is allowed in pharmaceuticals or foods can be used for the colorants, examples of which include blue dye no. 1, yellow dye no. 4, green dye no. 3, red dye no. 5, lake pigments, titanium dioxide, red cabbage pigment, red yeast pigment, purple sweet potato pigment, gardenia pigment and cochineal pigment.

Examples of the absorption promoters include surfactants such as higher alcohols, higher fatty acids and glycerin fatty acid esters.

Examples of the dissolution assistants include adipic acid, L-arginine, sodium benzoate, benzyl benzoate, esterified corn oil, ethanol, magnesium chloride, hydrochloric acid, olive oil, carmellose sodium, dry sodium carbonate, dilute hydrochloric acid, citric acid, sodium citrate, glycine, glycerin, glycerin fatty acid esters, geraniol, sesame oil, cellulose acetate phthalate, sodium salicylate, magnesium oxide, α-cyclodextrin, β-cyclodextrin, dibutylhydroxytoluene, tartaric acid, sucrose fatty acid esters, sodium hydroxide, sorbitan sesquioleate, sorbitan fatty acid esters, D-sorbitol, liquid D-sorbitol, soybean oil, soybean lecithin, sodium bicarbonate, sodium carbonate, medium-chain fatty acid triglycerides, triacetin, sorbitan trioleate, nicotinic amide, lactic acid, concentrated glycerin, cupronickel, hydroxypropyl methyl cellulose, castor oil, glacial acetic acid, glucose, propylene glycol, propylene glycol fatty acid esters, povidone, polyoxyethylene hydrogenated castor oil, polyoxyethylene (160) polyoxypropylene (30) glycol, polysorbate, polyvinyl alcohol, Macrogol, D-mannitol, isopropyl myristate, anhydrous ethanol, anhydrous citric acid, sorbitan monooleate, lauryl Macrogol, lidocaine, phosphoric acid, sodium hydrogen phosphate and potassium dihydrogen phosphate.

Examples of the stabilizers include benzoic acid, sodium benzoate and ethyl parahydroxybenzoate.

There are no particular limitations on the health food materials, and examples include Chinese herbal medicines (such as stomach-calming powder and poria powder with five herbs, meridian-warming decoction, warming and clearing decoction, ougikenchutou, astragalus middle-strengthening decoction, coptis decoction, pureraria decoction plus szeshwan lovage and magnolia flower, modified back to the spleen decoction, kamishoyosan, licorice, wheat and Chinese date decoction, balloon flower root decoction, back to the spleen decoction, areca seed decoction with nine herbs, schizonepeta and forsythia decoction, cassia twig decoction plus peony and rhubarb, keishikashakuyakuto, cassia twig plus dragon's bone and oyster shell decoction, cassia twig decoction, cassia twig plus ginseng decoction, cassia twig and tuckahoe pill, open the spleen decoction, cyperus and perilla leaf powder, five tiger decoction, powder for five kinds of stagnations, life-preserving kidney-qi pill, powder for five kinds of stranguria, combined minor blupeurum decoction and minor chest congestion decoction, blupeurum plus dragon's bone and oyster shell decoction, blupeurum cassia twig and dried ginger decoction, blupeurum and cassia twig decoction, blupeurum liver-clearing decoction, combined minor blupeurum decoction and pinellia and magnolia decoction, minor blupeurum decoction plus poria powder with five herbs, wild jujube seed decoction, yin nourishing and fire-eliminating decoction, cold limbs powder, four gentlemen decoction, four herbs decoction, roasted licorice decoction, peony and licorice decoction, ten strong tonic herbs decoction, antiphlogistic decoction with ten herbs, minor middle-strengthening decoction, minor blupeurum decoction, minor blue dragon decoction, wind dispersing powder, magnolia flower lung-clearing decoction, mystery decoction, north water god decoction, head-clearing divaricate saposhnikovia decoction, summer heat-clearing and qi-benefiting decoction, heat-clearing lotus seed decoction, lung-clearing decoction, channels-dredging and blood-activating decoction, rhubarb and licorice decoction, rhubarb and moutan bark decoction, major middle-strengthening decoction, major blupeurum decoction, major blupeurum decoction without rhubarb, major purgative decoction, major divaricate saposhnikovia decoction, for contusion decoction, stomach-regulating purgative decoction, uncaria powder, intestinal carbuncle decoction, umbellate fungus decoction, combined umbellate fungus decoction with four herbs decoction, dredging and dissipating powder, peach kernel purgative decoction, angelica antipruritis decoction, angelica middle-strengthening decoction, angelica and peony powder, angelica decoction, two vintage herbs decoction, goddess powder, ginseng decoction, ginseng nutrition decoction, pus-discharging powder and decoction, dwarf lilyturf decoction, kidney qi pill, pinellia and magnolia decoction, pinellia heart-purging decoction, white tiger plus ginseng decoction, tuckahoe decoction, combined tuckahoe decoction and pinellia and magnolia decoction, stomach-calming powder, stephania and astragalus decoction, divaricate saposhnikovia miraculous powder, middle-reinforcing and qi-benefiting decoction, ephedra decoction, ephedra, aconite and Manchurian wild ginger decoction, ephedra, apricot, licorice and gypsum decoction, hemp seed pill, fourstamen stephania decoction, liver-inhibiting powder, liver-inhibiting powder plus tangerine peel and pinellia tuber, six gentlemen decoction, instant effective powder, gentian liver-purging decoction, tuckahoe, licorice, dried ginger, schisandra, Manchurian wild ginger, pinellia and apricot decoction or six-ingredient pill with rehmannia), teas (such as green tea, genmai tea, macha green tea, sencha green tea, roasted hoji tea, baicha tea, jasmine tea, oolong tea, black tea, black tea, fermented flower tea, blue tea or white tea), natural herbs (such as Italian parsley, elecampane, olive, oregano, cardoon, chamomile, curry plant, catnip, caraway, Christmas rose, crimson clover, cornflower, common mallow, salad burnet, cotton lavender, cinnamon, jasmine, stevia, sage, European linden, scented geranium, St. John's wort, soapwort, Solomon's tool, thyme, tansy, chervil, chive, nasturtium, nutmeg, basil, honeysuckle, hyssop, flax, fennel, foxglove, black hollyhock, French marigold, betony, heliotrope, bergamot, hemp agrimony, rue, pot marigold, borage, white horehound, myrtle, mullein, marjoram, mint, yarrow, lavender, lady's bedstraw, lemon grass, lemon verbena, lemon balm, rose, rosemary, rocket, wild strawberry, wild pansy or forget-me-not), propolis, gingko nuts, kale drink and extracts thereof:

There are no particular limitations on the nutritional supplement materials, and examples include amino acids, metal ions, proteins, sugars, fatty acids, yeast extracts, vegetable extracts, fish and meat extracts, fruits and fruit extracts.

There are no particular limitations on the vitamins, and examples include vitamin A, vitamin B, vitamin C, vitamin D, vitamin E, vitamin K and derivatives thereof.

Examples of the fragrances include individual fragrances such as menthol, carbon, anethole, cineol, methyl salicylate, cinnamic aldehyde, eugenol, 3,1-menthoxypropane-1,2-diol, thymol, linalool, linalyl acetate, limonene, menthone, menthyl acetate, N-substituted-paramenthane-3-carboxamide, pinene, octyl aldehyde, citral, pulegone, carvyl acetate, anise aldehyde, ethyl acetate, ethyl butyrate, allylcyclohexane propionate, methyl anthranilate, ethylmethyl ethynyl glycidate, vanillin, undecalactone, hexanal, ethyl alcohol, propyl alcohol, butanol, isoamyl alcohol, hexenol, dimethylsulfide, cyclotene, furfural, trimethylpyrazone, ethyl lactate or ethyl thioacetate, natural fragrances such as peppermint oil, spearmint oil, anise oil, eucalyptus oil, wintergreen oil, cassia oil, clove oil, thyme oil, sage oil, lemon oil, orange oil, mentha oil, cardamom oil, coriander oil, mandarin oil, lime oil, lavender oil, rosemary oil, laurel oil, chamomile oil, caraway oil, marjoram oil, bay oil, lemon grass oil, origanum oil, pine needle oil, neroli oil, rose oil, jasmine oil, iris concrete, absolute peppermint, absolute rose or orange flower, and formulated fragrances such as strawberry flavoring, apple flavoring, banana flavoring, pineapple flavoring, grape flavoring, mango flavoring, butter flavoring, milk flavoring, mixed fruit flavoring or tropical fruit flavoring.

Examples of sweeteners include sodium saccharin, aspartame, stevioside, stevia extract, paramethoxycinnamic aldehyde, neohesperidyl dihydrochalcone and perillartine.

Examples of antiseptics include aminoethylsulfonic acid, benzoic acid, sodium benzoate, ethanol, sodium edetate, agar, dL-camphor, citric acid, sodium citrate, salicylic acid, sodium salicylate, phenyl salicylate, dibutylhydroxytoluene, sorbic acid, potassium sorbate, nitrogen, dehydroacetic acid, sodium dehydroacetate, 2-naphthol, saccharose, honey, isobutyl parahydroxybenzoate, ethyl parahydroxybenzoate, butyl parahydroxybenzoate, propyl parahydroxybenzoate, methyl parahydroxybenzoate, L-menthol and eucalyptus oil.

Examples of preservatives include benzoic acid, sodium benzoate, ethanol, sodium edetate, dry sodium sulfite, citric acid, glycerin, salicylic acid, sodium salicylate, dibutylhydroxytoluene, D-sorbitol, sorbic acid, potassium sorbate, sodium dehydroacetate, isobutyl parahydroxybenzoate, isopropyl parahydroxybenzoate, ethyl parahydroxybenzoate, butyl parahydroxybenzoate, propyl parahydroxybenzoate, methyl parahydroxybenzoate, propylene glycol and phosphoric acid.

Examples of antioxidants include citric acid, citric acid derivatives, vitamin C and derivatives thereof, lycopene, vitamin A, carotenoids, vitamin B and derivatives thereof, flavonoids, polyphenols, selenium, sodium thiosulfate, vitamin E and derivatives thereof, α-lipoic acid and derivatives thereof, pycnogenol, flavangenol, superoxide dismutase (SOD), glutathione peroxidase, glutathione-S-transferase, glutathione reductase, catalase, ascorbic acid peroxidase and mixtures thereof.

(Supplements)

The consumption of a composition containing the biopterin of the present invention directly, or consumption of a food or beverage containing the same can be considered for a nutritional supplement (supplement). Moreover, a composition containing the biopterin of the present invention can also be used as a health food or nutritional supplement food in the same manner.

(Foods)

In the case of foods, a composition containing biopterin may be consumed directly or a food or beverage containing the same may be consumed.

(Cosmetics)

Examples of cosmetics include beauty soaps, cleansers, soft peeling agents, scrubs, packs, facial wash, milky lotions, beauty wash, creams, foundation, hand cream, body cream, shampoo, rinse, lipstick, lip balm and eye shadow. These cosmetics may incorporate various types of additives normally used in cosmetic products. Examples of such arbitrary components, excluding essential components, include surfactants, alcohols, moisturizing agents, thickeners, antiseptics, antioxidants, chelating agents, pH adjusters, fragrances, pigments, colorants, ultraviolet absorbers/light scattering agents, vitamins, amino acids, pharmaceutically effective components and vegetable extracts. Furthermore, arbitrary components are not limited thereto.

Examples of oral compositions include toothpaste, tooth powder, liquid toothpaste, watery toothpastes, mouthwash, gum massage creams, oral sprays, intrabucccal tablets, liquid or paste-like topical coatings and chewing gum. Suitable components can be added to the oral composition of the present invention according to the purpose, type of composition and the like. In the case of a toothpaste, for example, in addition to calcium phosphate, calcium carbonate, aluminum hydroxide and magnesium carbonate, binders in the form of carrageenan or carboxycellulose, thickeners in the form of glycerin, ethylene glycol or sorbitan, surfactants or fragrances can be added.

(Animal Feeds)

A composition containing the biopterin of the present invention may be consumed directly or a food or beverage containing the same may be consumed in the case of pet foods and animal feeds. Moreover, a composition containing biopterin of the present invention can also be used as a health food or nutritional supplement food in the same manner.

Furthermore, there are two methods known among persons with ordinary skill in the art for producing the biopterin contained in the “biopterin composition” of the present invention. Although these consist of biological methods and chemical synthesis methods, biopterin produced by either of these methods can be used. In addition, BH4 may also be contained in the “biopterin composition” as necessary.

As has been described above, the use of a biopterin or biopterin composition in the form of an embodiment of the present invention as an alternative to BH4, which although having superior efficacy is unstable, difficult to handle and expensive, or in a form in which it is present with BH4 as necessary, makes it possible to provide a composition at least having effects equal to those of BH4 stably, in a form that is easy to use, and inexpensively.

### BRIEF DESCRIPTION OF THE DRAWINGS

FIG. 1 is a graph showing the amounts of biopterin of the embodiments that migrated to the liver three hours after single-dose oral administration to hph-1 mice;

FIG. 2 is a graph showing the amounts of biopterin of the embodiments that migrated to the kidneys 3 hours after single-dose oral administration to hph-1 mice;

FIG. 3 is a graph showing the amounts of biopterin of the embodiments that migrated to the brain 3 hours after single-dose oral administration to hph-1 mice;

FIG. 4 is a graph showing changes in the concentrations of BH4 in the blood at 48-hour intervals during oral administration of biopterin of the embodiments to hph-1 mice;

FIG. 5 is a graph comparing concentrations of BH4 in the brain 2.5 hours after continuous oral administration of biopterin of the embodiments and BH4 to hph-1 mice; and

FIG. 6 is a graph showing the amounts of biopterin of the embodiments that migrated to the urine 6 hours after single-dose oral administration to normal mice.

### BEST MODE FOR CARRYING OUT THE INVENTION

Although the following provides a more detailed explanation of the present invention by indicating examples thereof, the present invention is not limited to only these examples.

### Example 1

Mutated hph-1 mice having low levels of BH4 in the body due to a mutation in the regulatory region of GTP cyclohydrolase 1 (GTPCH1) gene, which is one of the genes involved in biosynthesis of BH4 (males, age 6 weeks, able to be acquired and produced by a person with ordinary skill in the art based on the description of Vernon C. Bode, et al., Genetics, 118, 299-305 (1988)), were used for the test mice. Biopterin was administered orally (suspended in 2% carboxymethyl cellulose solution (CMC solution)) at 10 mg/kg (amount of biopterin per 1 kg of body weight) to 4 test animals. 2 test animals were administered 2% CMC solution only for use as controls (comparative examples) thereof.

Each of the mice were autopsied 3 hours later, and the liver, kidneys and brain were excised and weighed followed by rapid-freezing in liquid nitrogen and storing at −80° C. The frozen organs were partially thawed followed by the addition of 5 volumes of 0.1 N HCl and homogenizing with a homogenizer. A half volume of acid-iodine solution (2% I₂ and 3% KI in 0.1 N HCl) or alkaline-iodine solution (2% I₂ and 3% KI in 0.2 N NaOH) was added to 0.1 ml of the homogenate followed by incubating for 1 hour at room temperature while shielding from light, adding 0.05 ml of 2.5% ascorbic acid-0.4 M perchloric acid solution, centrifuging (10,000×g, 10 min) and quantifying the entire amount of biopterins contained in the supernatant.

Quantification of biopterins was carried out in accordance with the method described in the prescribed literature (Fukushima, T., Nixon, J C., Anal. Biochem., 102, 176-188 (1980)). Namely, since biopterins are completely oxidized to biopterin in the case of iodine oxidation under acidic conditions, the entire amount of biopterins can be quantified. On the other hand, since only BH4 is oxidized to pterin while other biopterins are oxidized to biopterin in the case of iodine oxidation under alkaline conditions, the entire amount of biopterins other than BH4 can be quantified. Thus, the amount of BH4 can be quantified utilizing the difference between acidic conditions and alkaline conditions. Furthermore, analysis of biopterin was carried out by high-performance liquid chromatography (column: Fine-SIL C18T-5, eluent: aqueous 7% methanol solution, detection: fluorescence, excitation: 350 nm, detection: 450 nm).

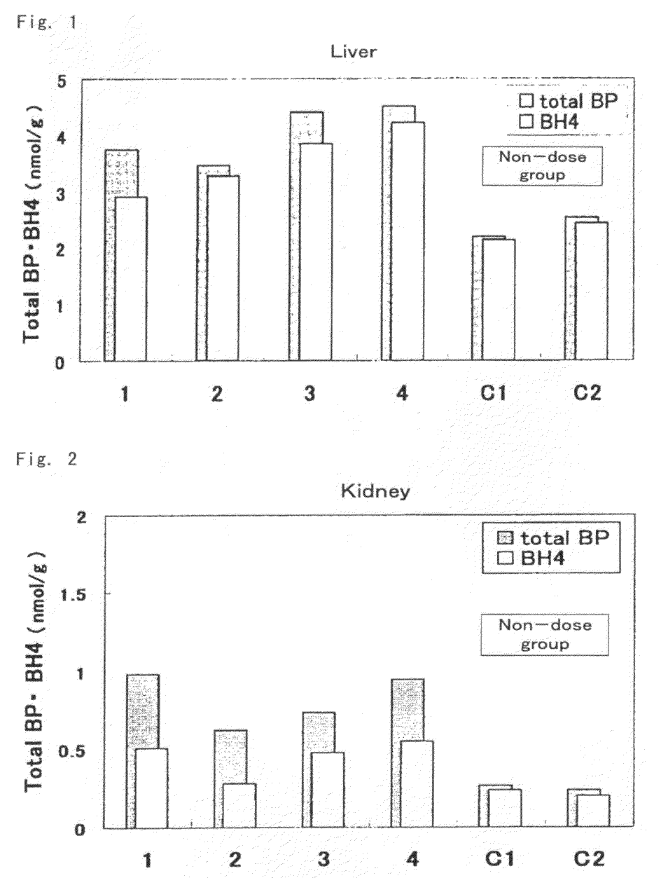

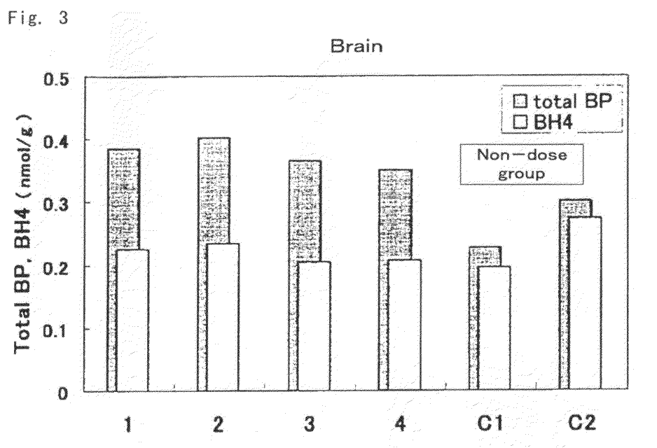

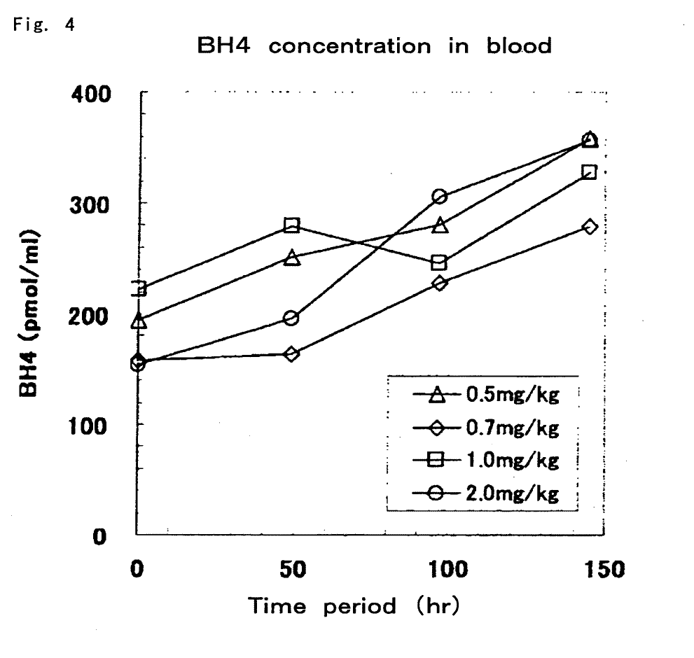

As a result of quantification, the amounts of biopterins increased considerably in each of the organs of test mice 1 to 4 (indicated with 1 to 4 in FIGS. 1 to 3) with the exception of the comparative examples (indicated with C1, C2 or non-dose group in FIGS. 1 to 3), the amounts of BH4 among the biopterins also increased significantly, and a high BH4 ratio was found to be maintained.

FIGS. 1, 2 and 3 are graphs showing the amounts of biopterins and BH4 in the liver, kidneys and brain, respectively, of hph-1 mice 3 hours after single-dose oral administration of the biopterin of the embodiments.

### Example 2

In Example 2, biopterin (0.5 mg/kg, 0.7 mg/kg, 1 mg/kg and 2 mg/kg) was orally administered for 6 consecutive days at 24-hour intervals to each of the hph-1 mice (age 10 weeks, males, total of 4 animals), and blood samples were collected every 48 hours. Quantification of blood BH4 concentrations was carried out in the same manner as Example 1 with the exception of using an acid-iodine solution (2% I₂ and 3% KI in 0.5 N HCl) or alkaline-iodine solution (2% I₂ and 3% KI in 1 N NaOH) in a composition exclusively for use with blood for a mixture 20 μL of blood and 80 μL of distilled water. As a result, as shown in FIG. 4, gradual increases in blood BH4 concentrations were observed at all dosages. On the other hand, in the case of having administered BH4, BH4 is known to exhibit behavior different from that during administration of biopterin, and has been reported to demonstrate a sharp peak 0.5 to 1 hours after administration followed immediately by a decrease in concentration and eventually returning to the original level about 4 hours later (Sawabe, K., et al., J. Pharmacol. Sci., 96, 124-133 (2004)).

FIG. 4 is a graph showing the concentrations of BH4 in the blood at 48-hour intervals during oral administration of biopterin of the embodiments for 6 consecutive days to hph-1 mice at 24-hour intervals.

### Example 3

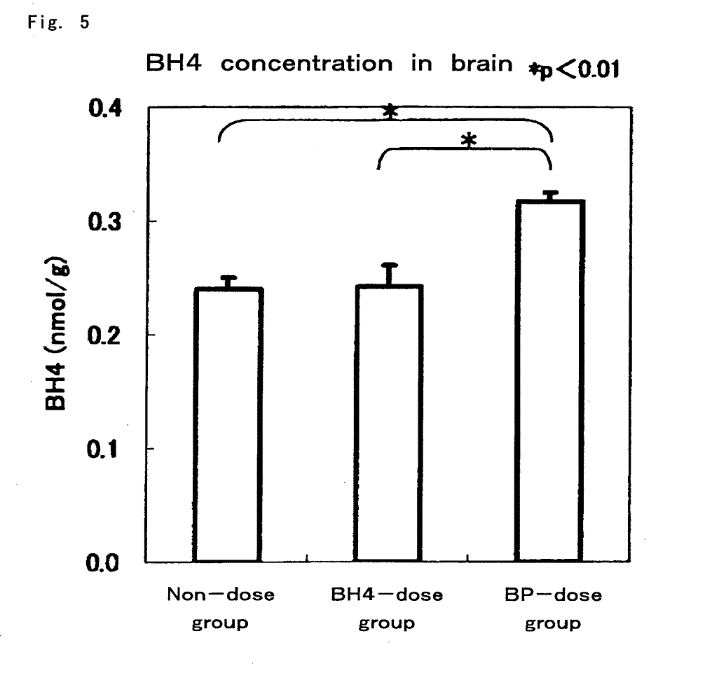

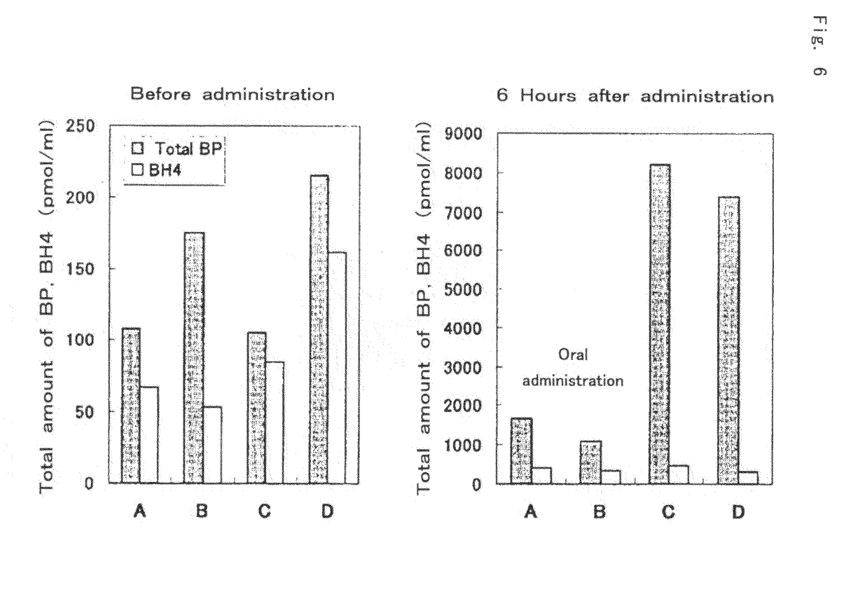

In Example 3, biopterin at 5 mg/kg or BH4 at 5 mg/kg, and 2% CMC solution only for a non-dose group, were orally administered to hph-1 mice (age 8 to 11 weeks, males, total of 12 animals) in groups of 4 animals each for 3 consecutive days at 24-hour intervals. The mice were biopsied 2.5 hours after the final dosing, their brains were excised and BH4 levels were quantified in the same manner as Example 1. As a result, as shown in FIG. 5, intracerebral BH4 concentrations demonstrated a remarkable increase only in the case of continuous oral administration of biopterin.

FIG. 5 is a graph comparing mean values of concentrations of BH4 in the brain 2.5 hours after the final dosing during oral administration of biopterin (BP) of the embodiments and BH4 to hph-1 mice for 3 consecutive days at 24-hour intervals.

### Example 4

In Example 4, an experiment was carried out in the same manner as Example 1 with the exception of the test mice, dosages, administration site (intraperitoneal administration) and sampling site. More specifically, biopterin was administered orally or intraperitoneally to normal C57-BL/6J mice (acquired from Japan SLC, Inc., age 8 to 10 weeks, males) in groups of 2 animals each at a dosage of 20 mg/kg, and the total amount of biopterins contained in urine samples collected 6 hours later were quantified. As a result, as shown in the graph of FIG. 6, biopterin levels increased considerably for either administration method, and BH4 levels also increased significantly.

FIG. 6 is a graph respectively showing the total amounts of biopterins and BH4 contained in urine collected before and 6 hours after single-dose oral administration or single-dose intraperitoneal administration of the biopterin of the embodiments. A, B, C and D respectively correspond to the test mice.

1. A biopterin composition which contains 100 μg or more of biopterin per 1 g of composition, and can be used as a pharmaceutical, functional food, supplement, food, cosmetic, veterinary drug, veterinary supplement or animal feed.

2. The biopterin composition according to claim 1, containing 1 mg or more of biopterin per 1 g of composition.

3. A method for increasing the concentration of tetrahydrobiopterin in a human or animal comprising administering to said human or animal on effective amount of biopterin to increase the concentration of tetrahydrobiopterin in the human or animal.

4. The method according to claim 3, having a therapeutic effect against malignant phenylketonuria or Segawa disease (dopa responsive dystonia) caused by tetrahydrobiopterin deficiency, or against phenylketonuria associated with inadequate activity of phenylalanine hydroxylase caused by the mutation thereof.

5. The method according to claim 3, having a protective action on vascular disorders caused by hypertension by promoting production of nitrogen monoxide in human or animal blood vessels and lowering blood pressure as a result of vasodilation due to the action of producing nitrogen monoxide.

6. The method according to claim 3, having a protective action on vascular disorders caused by the formation of active oxygen by promoting production of nitrogen monoxide in human or animal blood vessels and eliminating active oxygen by the action of producing nitrogen monoxide.

7. The method according to claim 3, having an effect of improving symptoms of bipolar disorder or schizophrenia, infantile autism, attention deficit hyperactivity disorder (ADHD), chronic fatigue syndrome, Parkinson's disease or Alzheimer's disease by promoting the formation of brain monoamines.

8. The method according to claim 3, having an effect of improving symptoms of depression, fatigue, arteriosclerosis, hypertension, hypercholesterolemia or impairment of vascular endothelial function by smoking.

9. The method according to claim 3, having an effect of improving symptoms of depression, fatigue, arteriosclerosis, hypertension or impairment of vascular endothelial function by hypercholesterolemia in animals.

10. The method according to claim 3, having sunburn preventive or whitening effects by inhibiting dermal melanocytes.

11. The method according to claim 3, demonstrating an effect that is mild and has superior sustainability as a result of using as a partial or complete alternative to tetrahydrobiopterin during administration thereof.

12. The method according to claim 3, demonstrating a superior increase in intracerebral tetrahydrobiopterin concentration as a result of using as a partial or complete alternative to tetrahydrobiopterin during administration thereof.

13. A pharmaceutical composition containing the biopterin composition according to claim 1.

14. A functional food, supplement or food containing the biopterin composition according to claim 1.

15. A veterinary drug, veterinary supplement or animal feed containing the biopterin composition according to claim 1.

16. A skin agent or cosmetic containing the biopterin composition according to claim 1.

17. A use of biopterin for producing the biopterin composition according to claim 1.
